(19) United States
(12) Patent Application Publication
(10) Pub. No.: US 2011/0070581 A1
(43) Pub. Date: Mar. 24, 2011

(54) Separation of Leukocytes

(76) Inventors: Amit Gupta, Somerville, MA (US); Alan Rosenbach, Worcester, MA (US); Aman Russom, Stockholm (SE); Kenneth T. Kotz, Auburndale, MA (US); Mehmet Toner, Wellesley, MA (US); Ronald G. Tompkins, Boston, MA (US)

(21) Appl. No.: 12/768,573

(22) Filed: Apr. 27, 2010

Related U.S. Application Data
(60) Provisional application No. 61/172,978, filed on Apr. 27, 2009.

(51) Int. Cl. C12Q 1/68; C12Q 1/04; C12N 5/078; C12N 5/0786

(52) U.S. Cl. 435/6; 435/34; 435/325

(57) ABSTRACT

Leukocytes (e.g., neutrophils, monocytes and/or lymphocytes) can be captured and separated from blood by removing platelets using a spiral channel, followed by capturing individual leukocyte types in a series of cell capture channels having leukocyte binding moieties. Accordingly, various microfluidic-based cell affinity chromatography methods can be used to separate leukocytes from whole blood.

### CROSS-REFERENCE TO RELATED APPLICATION

This application claims the benefit of U.S. Provisional Patent Application Ser. No. 61/172,978, filed on Apr. 27, 2009, the entire contents of which are hereby incorporated by reference.

### TECHNICAL FIELD

This disclosure relates to the separation of leukocytes from samples, e.g., whole blood samples.

### BACKGROUND

Techniques for collecting, detecting and analyzing leukocytes from biological samples are useful for a variety of applications. Transcriptome analysis of circulating peripheral leukocytes from critically ill patients can provide a wealth of new information, as well as diagnostic and prognostic data, which when acquired in a timely manner can very likely save the patient's life. Disease specific markers from the patient can be used to assess the progress of the disease, as well as the therapeutic outcome.

But in spite of the tremendous progress that has been achieved in gene expression profiling in basic medical research and clinical science, there remains a need for additional techniques for studying the biological basis of complex human diseases. The development of improved analytical technologies, such as automated RNA isolation and techniques to isolate cell populations by cell capture and release, can lead to adoption of gene expression analysis in an everyday hospital clinical setting. Improvements in these techniques can include reduction in technician time, and minimizing the sample size and reagent use in technologies such as oligonucleotide microarrays. One of the major challenges for high-throughput genomics technology becoming more prevalent clinically has been a need to reduce the amount of blood handling and separation of cells required in the hospital setting. There is a need to develop a point-of-care method that will have an automated and integrated approach, to isolate and enrich specific leukocyte subpopulations using a single source of blood.

The use of microfabrication technology to miniaturize methods and means for analysis in the laboratory has led to the creation of an area known as micro total analysis systems (microTAS) or simply as “lab-on-a-chip.” The main attraction of this burgeoning area is its focus on methods that can perform real-time analysis, with a minimum use of analytes and reagents (usually in the amounts of microliters and down to picoliters), and having the entire operation performed in an automated (or semi-automated) and economical manner.

### SUMMARY

This disclosure relates to the separation of leukocytes from blood samples, e.g., whole blood samples, for example by passing a blood sample through a fluid flow path containing a spiral channel and a series of cell capture channels on an integrated microfluidic device. The invention is based on the discovery that neutrophils, monocytes, and lymphocytes can be captured and separated with a purity of at least about 90% by first removing platelets using a spiral channel, followed by capturing individual leukocyte types in a series of cell capture channels having leukocyte binding moieties.

Methods for capturing and detecting one or more leukocyte cells from a sample can include passing the sample through a spiral fluid flow channel to separate the sample into a platelet enriched stream and a leukocyte enriched stream. Preferably, passing the sample through the spiral channel does not alter the activation state of the leukocyte cell population. The leukocyte enriched stream can be passed through one or more cell capture channels including binding moieties selected to bind to one or more leukocyte cells. The dimensions of each cell capture channel and the flow rate can be selected to capture and retain at least a portion of a first leukocyte cell population within a cell capture channel, thereby reducing the first leukocyte cell population in the leukocyte enriched stream. For example, the leukocyte cell population can be selected from the group consisting of monocytes, neutrophils and lymphocytes. Separate types of leukocytes can be captured in separate cell capture chambers. The captured first leukocyte cell population can be detected, for example, by lysing the first leukocyte population within the first cell capture channel and measuring RNA obtained therefrom. The cell capture process can be repeated for different types of leukocytes by passing the fluid through cell capture channels configured to capture different types of leukocytes (e.g., having different binding moieties, different flow rates and/or different dimensions). In this manner, a plurality of cell capture channels, each channel retaining a separate population of captured leukocyte cells that contains at least about 90%-95% of a single type of leukocyte. Highly purified discrete leukocyte cell populations can be obtained in cell capture channels for separate subsequent analysis. As a result, the amount of each type of leukocyte cell present in the original sample can be determined using the integrated microfluidic devices and methods disclosed herein.

For example, integrated microfluidic devices described herein can separate and isolate the three major circulating peripheral leukocyte sub-populations (i.e., neutrophils, lymphocytes and monocytes), using a single source of whole blood (volume amount<1.0 mL) from acute critically ill patients. Preferred methods of leukocyte separation using the integrated microfluidic devices disclosed herein can provide leukocyte separation and analysis in less than 30 minutes, including the time period from introducing the blood sample into the chip to obtaining the lysate of the captured cells. The integrated microfluidic devices and methods disclosed herein can permit each leukocyte cell subtype to be isolated using the same method.

In one aspect, the invention features methods for capturing and detecting a leukocyte sub-population from a blood sample. The method includes providing a sample including a first leukocyte cell population, a second leukocyte cell population and a platelet cell population; passing the sample through a spiral channel to separate the sample into a platelet enriched stream and a leukocyte enriched stream; passing the leukocyte enriched stream through a first cell capture channel at a first rate to capture and retain at least a portion of the first leukocyte cell population within the first cell capture channel, thereby reducing the first leukocyte cell population in the leukocyte enriched stream; and detecting the first leukocyte cell population retained in the first cell capture channel.

In certain embodiments, the methods can further include lysing at least a portion of the first leukocyte cell population retained in the first cell capture channel; and detecting the first leukocyte cell population by measuring the amount RNA (or protein) obtained from the first cell capture channel after lysing the portion of the retained first leukocyte cell population.

In these methods the RNA quality, expressed as RNA integrity number (RIN), can be at least about 7.5, and the total amount of RNA can be at least about 5.0 ng or greater. In certain embodiments, the first leukocyte cell population in the leukocyte enriched stream is reduced by at least about 90% while passing through the first cell capture channel, and the first leukocyte cell population and the second leukocyte cell population can be different types of leukocyte cells selected from the group consisting of monocytes, neutrophils, and lymphocytes. In some embodiments, the sample is passed through a spiral channel without altering the activation state of the first leukocyte cell population, e.g., the first leukocyte cell population can be inactive.

In some embodiment the first leukocyte cell population comprises “a majority of neutrophils,” which means that more than half of the leukocytes in the first leukocyte cell population are neutrophils. In some embodiments the second leukocyte cell population can comprise a majority of monocytes.

In certain embodiments, the methods can further include passing a first leukocyte depleted stream obtained from the first cell capture channel through a second cell capture channel at a second rate to capture and retain at least a portion of the second leukocyte cell population within the second cell capture channel, thereby reducing the second leukocyte cell population in the first leukocyte depleted stream; and detecting the second leukocyte cell population retained in the second cell capture channel. For example, in these methods the first leukocyte population can comprise a majority of neutrophils and the second leukocyte cell population can comprise a majority of monocytes, and the first rate and the second rate can be substantially equal and the volume of the first cell capture channel can be different from the volume of the second cell capture channel.

In other embodiments, the methods can further include passing a second leukocyte depleted stream obtained from the second cell capture channel through a third cell capture channel at a third rate to capture and retain at least a portion of a third leukocyte cell population within the third cell capture channel, reducing the third leukocyte cell population in the second leukocyte depleted stream; and detecting the third leukocyte cell population retained in the third cell capture channel. In these methods the first leukocyte population can comprise a majority of neutrophils, the second leukocyte cell population can comprise a majority of monocytes, and the third leukocyte population can comprise a majority of lymphocytes.

In another aspect, the invention features methods for capturing multiple leukocyte sub-populations from a blood sample. The methods include providing a sample including a first leukocyte cell, a second leukocyte cell, and a platelet cell population; passing the sample through a spiral channel to separate the sample into a platelet enriched stream and a leukocyte enriched stream without altering the activation state of the first leukocyte cell; and passing the leukocyte enriched stream through a first cell capture channel to remove the first leukocyte cell from the leukocyte enriched stream.

These methods can further include retaining the first leukocyte cell within the first cell capture channel; lysing the first leukocyte cell retained in the first cell capture channel; and detecting the first leukocyte cell by measuring the amount of RNA (or protein) obtained from the first cell capture channel after lysing the portion of the retained first leukocyte cell population.

In certain embodiments, the leukocyte enriched stream can further include a second leukocyte cell that is different from the first leukocyte cell; and the first leukocyte cell and the second leukocyte cell can be selected from the group consisting of: monocytes, neutrophils, and lymphocytes.

These methods can further include passing a second leukocyte depleted stream obtained from the first cell capture channel through a second cell capture channel at a second rate to capture and retain a second leukocyte cell population within the second cell capture channel, thereby reducing the second leukocyte cell population in the first leukocyte depleted stream; and detecting the second leukocyte cell population retained in the second cell capture channel.

In another aspect, the invention features methods for capturing and detecting multiple leukocyte sub-populations from a blood sample. These methods include providing a blood sample having a first leukocyte cell population, a second leukocyte cell population, and a platelet cell population; passing the sample through a spiral channel to separate the sample into a platelet enriched stream and a leukocyte enriched stream without altering the activation state of the first leukocyte cell population; passing the leukocyte enriched stream through a first cell capture channel at a first rate to capture and retain at least a portion of the first leukocyte cell population within the first cell capture channel, thereby reducing the first leukocyte cell population in the leukocyte enriched stream; detecting the first leukocyte cell population retained in the first cell capture channel; passing a first leukocyte depleted stream obtained from the first cell capture channel through a second cell capture channel at a second rate to capture and retain at least a portion of the second leukocyte cell population within the second cell capture channel, thereby reducing the second leukocyte cell population in the first leukocyte depleted stream; detecting the second leukocyte cell population retained in the second cell capture channel; passing a second leukocyte depleted stream obtained from the second cell capture channel through a third cell capture channel at a third rate to capture and retain at least a portion of a third leukocyte cell population within the third cell capture channel, reducing the third leukocyte cell population in the second leukocyte depleted stream; and detecting the third leukocyte cell population retained in the third cell capture channel.

In these methods, the first leukocyte population can comprise a majority of neutrophils, the second leukocyte cell population can comprise a majority of monocytes, and the third leukocyte population can comprise a majority of lymphocytes.

By “binding moieties” is meant a molecule that specifically binds to an analyte (e.g., a cell). Binding moieties include, for example, antibodies, antibody fragments (e.g., Fc fragments), aptamers, receptors, ligands, antigens, biotin, avidin, metal ions, chelating agents, coordination complexes, nucleic acids, carbohydrates, MHC-peptide monomers, tetramers, pentamers or other oligomers.

By “cell surface marker” is meant a molecule bound to a cell that is exposed to the extracellular environment. The cell surface marker can be a protein, lipid, carbohydrate, or some combination of the three. The term “cell surface marker” includes naturally occurring molecules, molecules that are aberrantly present as the result of some disease condition, or a molecule that is attached to the surface of the cell.

By “lysis” is meant disruption of the cellular membrane. For the purposes of this disclosure, the term “lysis” is meant to include complete disruption of the cellular membrane (“complete lysis”), partial disruption of the cellular membrane (“partial lysis”), and permeabilization of the cellular membrane.

The term “chamber” is meant to include any designated portion of a microfluidic channel, e.g., where the cross-sectional area is greater, less than, or the same as channels entering and exiting the chamber.

As used herein, the phrase “leukocyte depleted stream” refers to a fluid having a reduced content of one or more types, without specificity, of leukocytes relative to a particular reference point (e.g., relative to the content of the same types of leukocytes prior to entering a microfluidic device or portion thereof).

As used herein, the phrase “first leukocyte depleted stream” refers to a fluid having a reduced content of a first particular type of leukocyte (e.g., a neutrophil-depleted stream), as opposed to a “leukocyte depleted stream,” relative to a particular reference point (e.g., relative to the number of neutrophils in the sample prior to entering a microfluidic device or portion thereof). In comparison, a “second leukocyte depleted stream” is a fluid with a reduced content of a second type of leukocyte (e.g., a monocyte depleted stream).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1A is top view of a first integrated microfluidics device.

FIG. 1B is a schematic of a second integrated microfluidics device.

FIG. 1C is a schematic of a third integrated microfluidics device.

FIG. 1D is a schematic of a spiral channel portion of an integrated microfluidics device.

FIG. 2 is a flow chart showing the basic operating steps of the microfluidics device shown in FIG. 1A.

FIG. 3A is a schematic of a spiral channel for separating analytes based on size.

FIG. 3B is an image of particles within a spiral channel.

FIGS. 4A and 4B are schematic diagrams of forces acting on fluid in the spiral channel shown in FIG. 3A.

FIG. 5 is a graph showing the particle count as a function of particle size and position at the outlet of the spiral channel shown in FIG. 3A.

FIG. 6 is a graph showing the normalized platelet count and white blood cell count corresponding to various whole blood dilutions.

FIG. 7 is a flow chart showing a testing protocol using an integrated microfluidics device.

FIGS. 8A and 8B are images obtained from FACS (fluorescent activated cell sorting) of leukocytes.

### DETAILED DESCRIPTION

Using the new systems and methods described herein, leukocytes (e.g., neutrophils, monocytes and lymphocytes) can be easily separated from blood samples and captured in discrete cell binding channels with a purity of at least about 90% in each cell binding channel. Preferably, platelets are first removed from the blood sample using a spiral channel, and then individual leukocyte cell types are captured and detected in a series of cell capture channels having different leukocyte binding moieties, such as antibodies. As described herein, integrated microfluidic devices including a spiral channel in fluid flow communication with one or more cell binding/capture channels can be used to separate leukocytes from blood samples.

### Microfluidic Systems

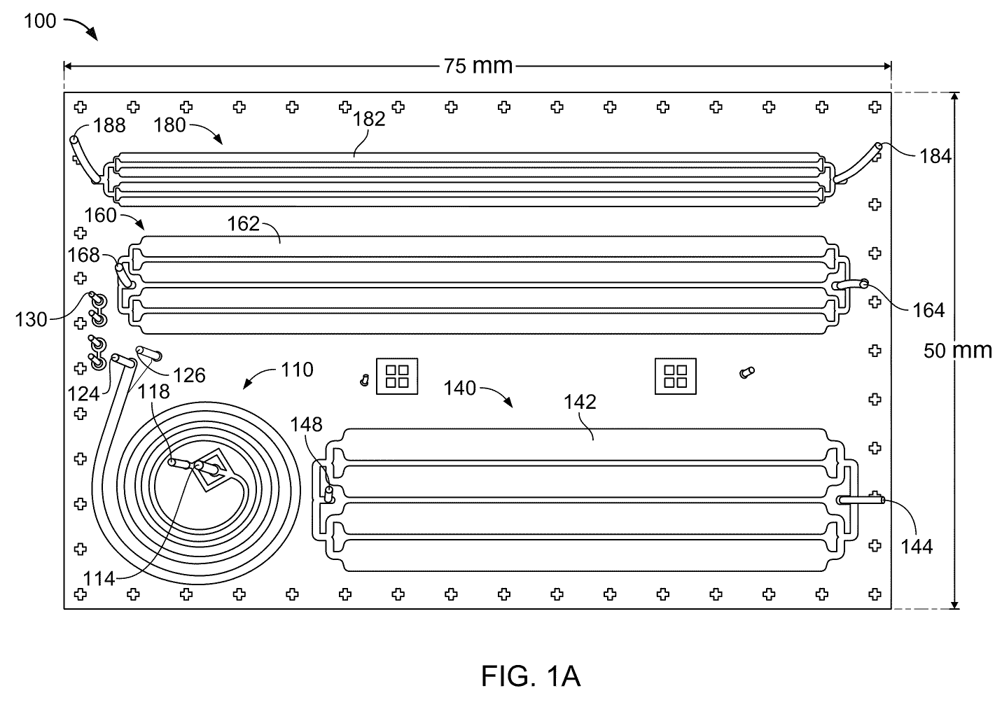

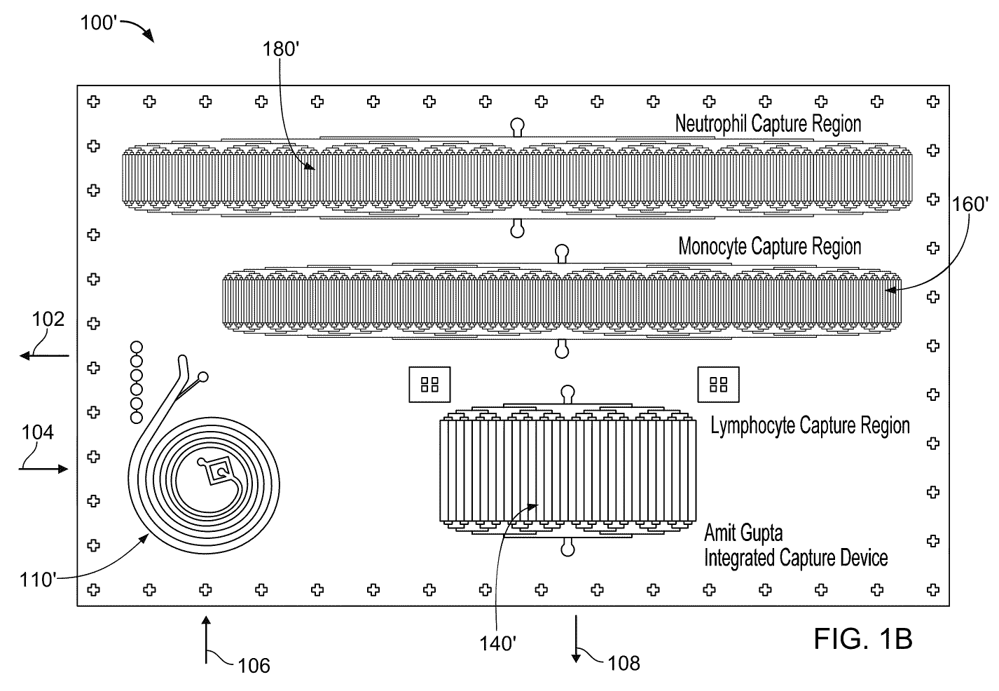

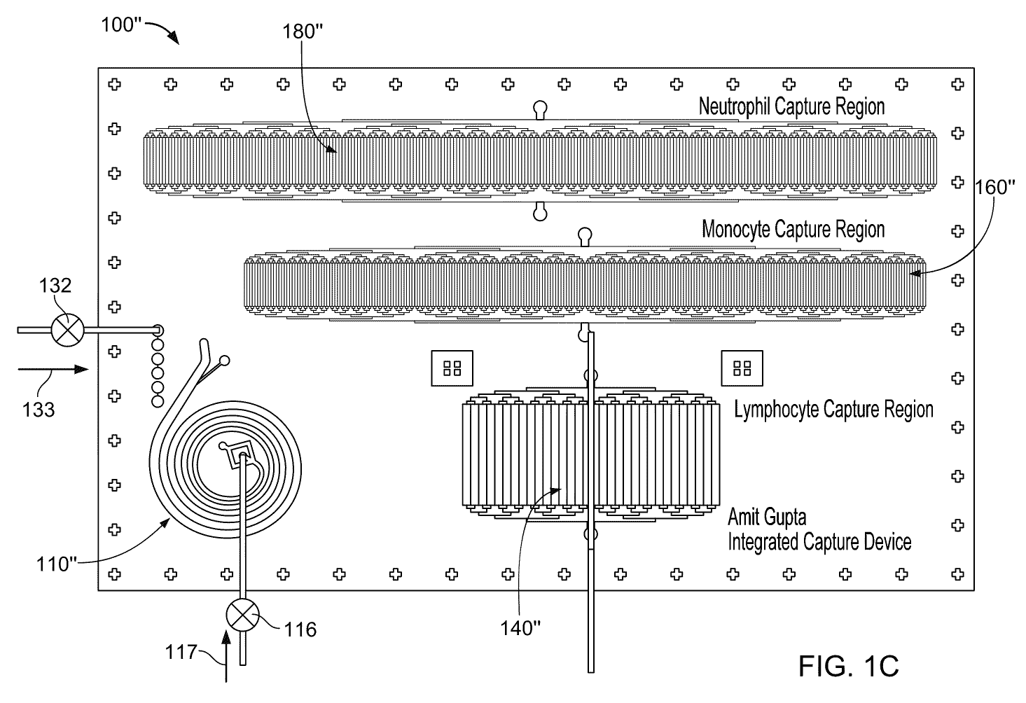

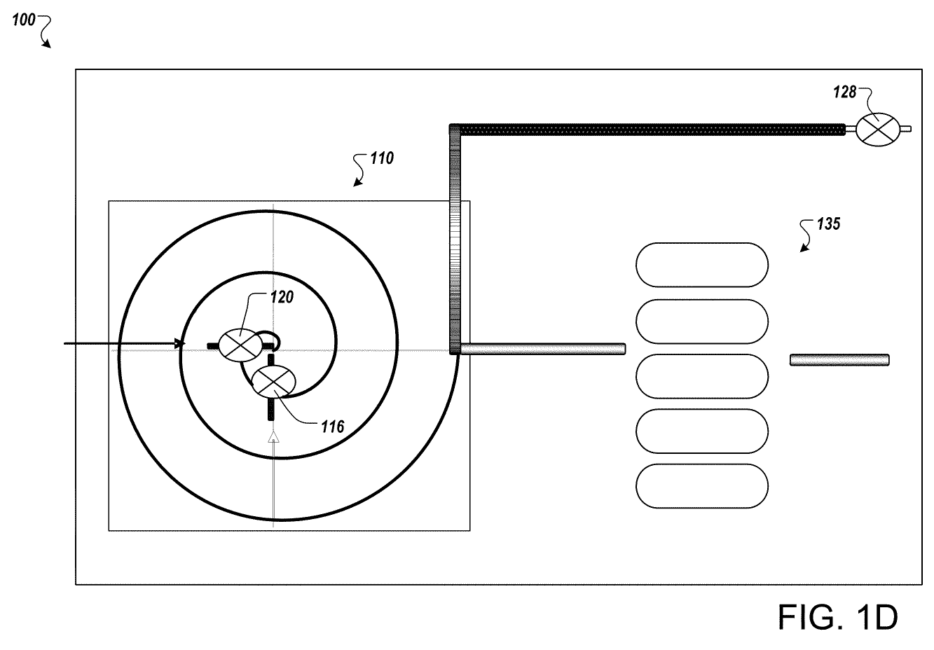

FIG. 1A shows a first integrated microfluidic device 100 for capturing and detecting a leukocyte sub-population from a blood sample. The integrated microfluidic device 100 includes a series of four sequentially connected fluid flow channel arrays forming a fluid flow path through the integrated microfluidic device 100: a spiral channel 110 that applies differential inertial focusing to the sample, a first cell capture channel 140, a second cell capture channel 160, and a third cell capture channel 180. The arrangement of components in FIG. 1A is exemplary, and other integrated microfluidic devices can contain other fluid flow path configurations (e.g., multiple or different spiral channels, and more or fewer cell capture channels).

The spiral channel 110 is configured to separate two or more components of a fluid sample based on relative mass, such as platelets and leukocytes. FIG. 1A shows that spiral channel 110 can have a non-uniformly varying radius of curvature, as well as width. For example, the spiral channel 110 shown in FIG. 1A has a channel height of 50 micrometers and a footprint area of 2.5 cm×1.5 cm. There are two inputs ports to the spiral channel 110, one (118) for the buffer (e.g., injected at 2500 μL/min), and a second input port 114 for a fluid sample (e.g., whole blood injected at 50 μL/min). The spiral channel 110 has two outputs: a leukocyte enriched (platelet depleted) output 126 that is preferably in fluid flow communication with an inlet to at least one cell capture channel, and the platelet enriched (leukocyte depleted) fluid output 124. In the spiral channel 110, the leukocyte enriched output can flow out of the spiral channel 110 at a rate of about 180-200 microliters/min and a platelet enriched fluid stream at a rate of about 2,300 μL/min (sent to a collection or waste vessel).

In certain embodiments, the spiral channel can be an asymmetrically curved channel described in U.S. patent application publication number US 2009/0014360, filed Apr. 16, 2008, portions of which pertaining to the separation of particles or cells based on relative mass within an asymmetrically curved channel are incorporated herein by reference.

The platelet enriched (leukocyte depleted) stream from first outlet 124 of the spiral channel 110 is injected into a hydrodynamic resistance balance 130 used to balance the hydrodynamic resistance provided to fluid exiting through the second outlet 126 by the series of cell capture channels (140, 160, 180). The outlet of the hydrodynamic resistance balance 130 can be a waste stream to remove the platelet enriched stream from the integrated microfluidic device 100.

The various components of the integrated system are connected by conduits, e.g., thin tubing attached externally to the ports or microchannels cut into the substrate between the various outlets and inlets, as follows. First, the leukocyte enriched stream from the second outlet 126 is directed or injected into the inlet 148 of the first cell capture channel 140. The leukocyte enriched stream passes through the first cell capture channel 140 and a first leukocyte depleted stream exits the first cell capture channel 140 at the outlet 144. The first leukocyte depleted stream from the outlet 144 enters a conduit that directs the flow to the second cell capture channel 160 through inlet 164. At least a portion of a second leukocyte cell population is retained within the second cell capture channel 160 as the first leukocyte depleted stream passes through the second cell capture channel 160 and a second leukocyte depleted stream exits the second cell capture channel 160 at the outlet 168.

The second leukocyte depleted stream from the outlet 168 passes into a conduit to enter the third cell capture channel 180 through inlet 188. At least a portion of a third leukocyte cell population is retained within the third cell capture channel 180 as the second leukocyte depleted stream passes through the third cell capture channel 180 and a third leukocyte depleted stream exits the third cell capture channel 180 at the outlet 184. The third leukocyte depleted stream can be collected in a collection vessel for subsequent analysis or disposal.

The integrated microfluidic device 100 can have two outputs: the platelet enriched (leukocyte depleted) stream exiting from the hydrodynamic resistance output 130, and the third leukocyte depleted stream from the third cell capture chamber outlet 184. Leukocytes can be captured from about 500 microliters of whole blood within about 10 minutes. In some examples, the integrated microfluidic device 100 is a single microfluidic platform for the isolation and enrichment of the three major leukocyte sub-populations: monocytes, neutrophils, and lymphocytes, using less than about 1 mL of blood. The cell capture channels can preferably selectively bind one of monocytes, neutrophils, or lymphocytes with at least about 95% purity. Examples of alternative microfluidic device configurations are shown in FIGS. 1B-1C.

FIG. 1B shows a second integrated microfluidic device 100′ with a spiral channel 110′, a first cell capture channel 140′, a second cell capture channel 160′ and a third cell capture channel 180′ joined in series in fluid flow communication. The second integrated microfluidic device 100′ is identical to the integrated microfluidic device 100 discussed with respect to FIG. 1A, except as described herein. Each of the cell capture channels (110′, 140′, 180′) of the second integrated microfluidic device 100′ are configured as a series of individual parallel channels joined to a common inlet and outlet, and oriented perpendicularly to the corresponding parallel channels (110, 140, 180) in the integrated microfluidic device 100 in FIG. 1A. The integrated microfluidic device 100′ can have two inputs: the blood sample (arrow 106) and a solution of 1% bovine serum albumin (BSA) in 1×PBS (arrow 104) are injected into the spiral channel 110′. The integrated microfluidic device 100′ can have two outputs: the platelet enriched (leukocyte depleted) stream exiting from the hydrodynamic resistance output (arrow 102), and the third leukocyte depleted stream from the third cell capture chamber outlet (arrow 108).

FIG. 1C shows a third integrated microfluidic device 100″ with a spiral channel 110″, a first cell capture channel 140″ (neutrophil capture), a second cell capture channel 160″ (monocyte capture) and a third cell capture channel 180″ (lymphocyte capture). The third integrated microfluidic device 100″ is identical to the integrated microfluidic device 100 discussed with respect to FIG. 1A, except as described herein. Each of the cell capture channels (110″, 140″, 180″) of the third integrated microfluidic device 100″ are configured as a series of individual parallel channels oriented perpendicularly to the corresponding parallel channels (110, 140, 180) in the integrated microfluidic device 100 in FIG. 1A.

The third integrated microfluidic device 100″ can have two inputs: the blood sample (arrow 117) injected through a first valve 116 and a solution of 1% BSA in 1×PBS (arrow 133) injected into the spiral channel 110″ through a second valve 132. The integrated microfluidic device 100″ can have two outputs, as described with respect to the second integrated microfluidic device 100′: the platelet enriched (leukocyte depleted) stream exiting from the hydrodynamic resistance output, and the third leukocyte depleted stream from the third cell capture chamber outlet.

FIG. 1D is a detailed view of the inlet portion of a spiral channel 110, as shown in FIG. 1A, showing the relative position of a first valve 120 to regulate injection of the buffer (e.g., BSA/PBS) into the spiral channel 110 to “prime” the fluid flow path, and a second valve 116 to regulate the subsequent injection of a blood sample (e.g., 0.5 mL volume) into the spiral channel 110. Valve 128 is a valve to control the flow of platelet enriched, leukocyte depleted fluid out of the spiral device. Valve 128 allows control of the washing buffer flow through the device, to be sent only though the capture channels, once the requisite amount of blood is flown through the capture device, and the washing of the chip to remove unbound cells is commenced. The buffer solution used to dilute the blood inside the spiral can also be used to wash off the cells. The buffer to wash can be injected through the spiral first and then to the capture channels. Using valve 128 at output of the platelet enriched fluid from the spiral, all the buffer is diverted to the capture channels. This allows for the device to be automated when running the device. Capture channels 135 (e.g., the cell capture channels 140, 160, 180 described in connection with FIG. 1A) are also shown schematically in FIG. 1D.

### Methods of Use

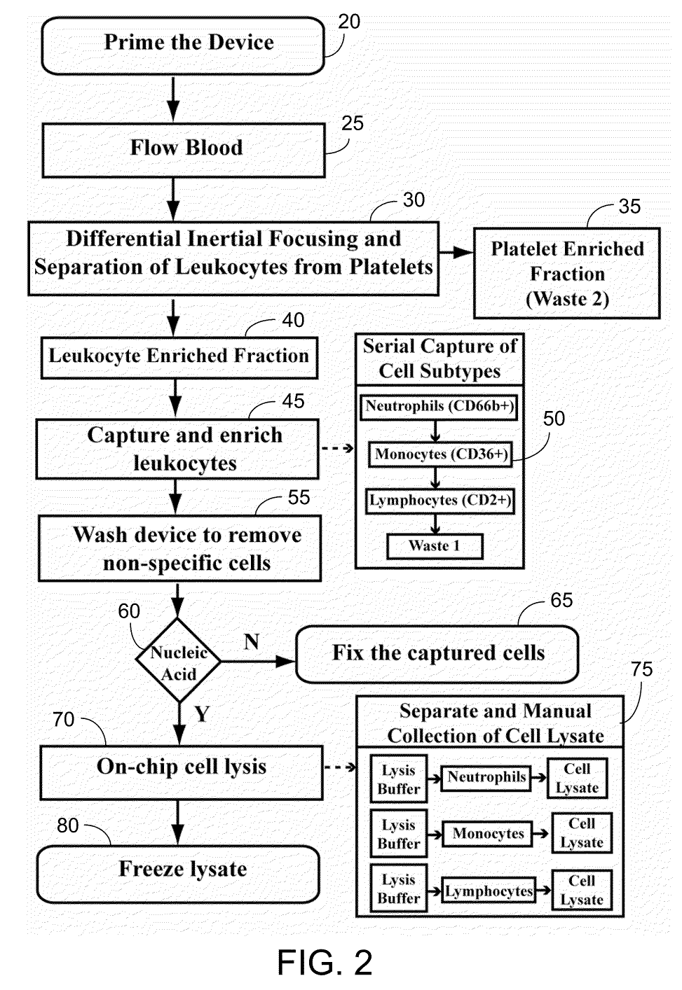

As shown in FIG. 2, in operation, the user can first inject a phosphate buffered saline (PBS, 1×) into the spiral channel 110 at a first inlet or input port 118 (e.g., at 2,500 microliters/min) to establish fluid flow through the integrated microfluidic device 100 fluid flow path (“priming” the flow path, step 20). After priming the fluid flow path, a blood sample can be injected at second inlet or input port 114 to initiate the capture of various leukocyte populations within the integrated microfluidic device 100 (step 25). The blood sample can be injected at a suitable flow rate (e.g., 50 microliters/min) into the spiral channel 110 through the second inlet or input port 114. The blood sample can include platelets, first leukocytes (e.g., neutrophils), second leukocytes (e.g., monocytes) and third leukocytes (e.g., lymphocytes).

The blood sample is passed through the spiral channel 110, to undergo differential inertial focusing and to produce a platelet enriched (leukocyte depleted) stream exiting the spiral channel through first outlet 124 (step 35) and a leukocyte enriched (platelet depleted) stream exiting the spiral channel through the second outlet 126 (step 30). The leukocyte enriched fraction is directed (step 40) to the cell capture channels (e.g., channels 140, 160, 180 in FIG. 1A) (step 45) and a specific type of leukocyte (e.g., neutrophils, monocyte, or lymphocyte) is removed in each channel (step 50).

Thereafter, the device is washed to remove non-specific cells (step 55) and the user needs to determine whether or not to extract nucleic acids from the cells (step 60). If not, then the captured cells can be fixed using standard techniques (step 65). If so, then on-chip cell lysis can be done (step 70). After the lysis step, one can separate and manually or automatically collect the cell lysate for different types of leukocytes (step 75) or freeze the entire lysate (step 80) for later analysis.

### Differential Inertial Focusing

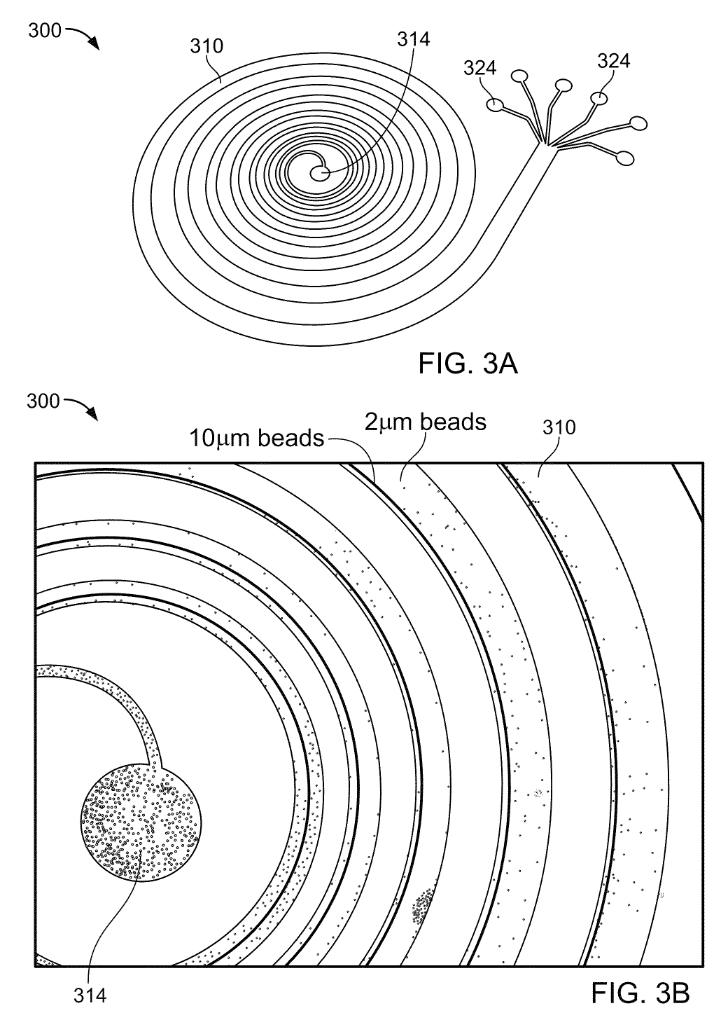

The devices described herein all include a spiral channel that is designed to apply differential inertial focusing to the blood sample. FIG. 3A shows a detailed view of a spiral channel 300 having an input 314, a curved channel 310 and a series of outputs 324 positioned to separate analytes in the sample based on mass and flow trajectory. FIG. 3B is a fluorescent image of a spiral channel 300 showing spatial separation of 10 micrometer beads and 2 micrometer beads originating from a source 314 in the center of the spiral channel 300. The flow rate in FIG. 3B is about 1.5 mL/min.

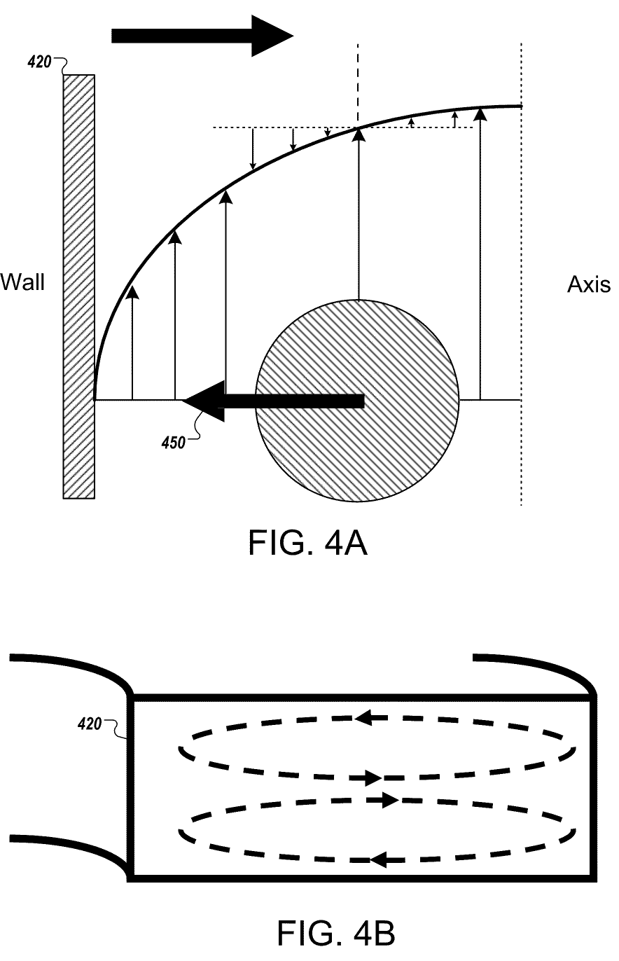

FIGS. 4A and 4B are schematic diagrams illustrating two forces that act to separate analyte components based on relative mass within the spiral channel 110: inertial lift forces (FIG. 4A), and Dean drag forces (FIG. 4B). Referring to FIG. 4A, shear-gradient-induced inertia 450 is directed down the shear gradient toward the wall 420 and the wall-induced inertia pushes particles away from the stationary wall. These forces are a function of various parameters such as flow rate, particle radius, channel dimension, and radius of curvature of curved channel. Dean flow (FIG. 4B) is a secondary rotational flow caused by inertia of the fluid itself. These forces cause the differential inertial focusing of any particles, e.g., cells, within the spiral channel 300.

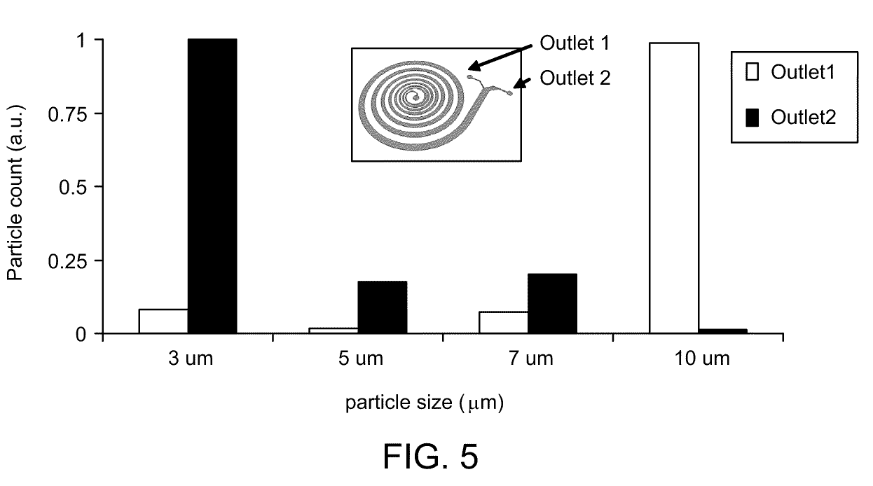

FIG. 5 is a histogram showing the number of particles as a function of particle size that were isolated at two different outlets from the spiral channel 300. Notably, the smallest (3 micrometer) particles were mostly localized from outlet 1 on one side of the curved channel 310, while largest (10 micrometer) particles were mostly localized from outlet 2, on the opposite side of the curved channel 310 at the outlet 2. Thus, the spiral channel 300 was able to positionally resolve particles of different sizes from 3-10 micrometers, depending on where the output was collected.

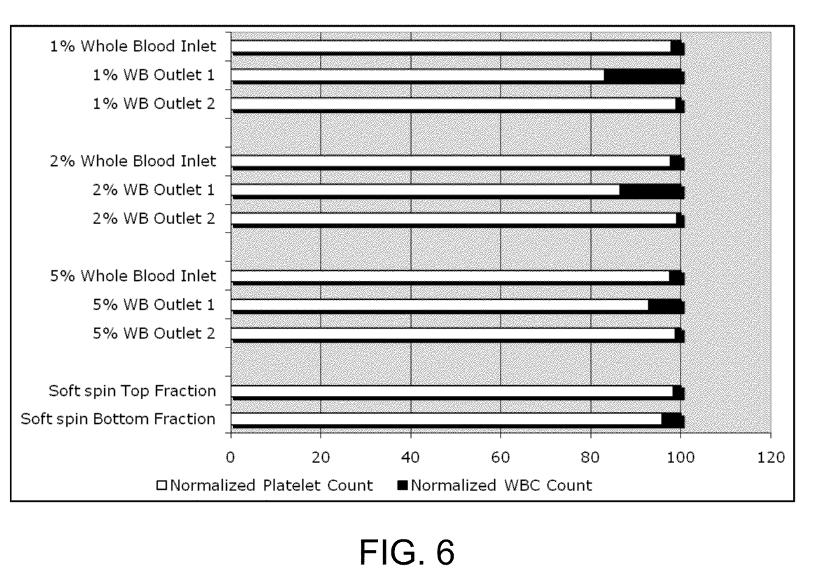

Blood can be diluted prior to injection into the spiral device, for example to reduce the particle to particle interaction, which would not allow for focusing of the cells within the spiral device. FIG. 6 is a graph showing normalized platelet count, normalized white blood cell count obtained at different whole blood (“WB”) dilutions from the spiral channel separator. When on-chip dilution is used in the spiral, the dilution of the blood can be performed inside the spiral. For example, 1% dilution corresponds to 25 μL/min flow of blood and 2475 μL/min of the buffer. The measurements of the cell fractions in FIG. 6 were done using a flow cytometer. The whole blood leukocyte to platelet ratio was about 0.023.

### Isolating Different Types of Leukocytes

The configuration of each cell capture channel (110, 140, 160) can be selected to permit retention of different leukocytes in each channel at a constant fluid flow rate through the fluid flow path. Each cell capture channel can include a binding moiety specific for one or more leukocyte cells, allowing these cells in a sample to bind to the binding moiety. Often, multiple leukocyte cell types can bind to a single binding moiety. Two or more leukocyte cell types of differing relative size bound to a binding moiety in a cell capture channel can be separated by applying a shear stress to the bound leukocyte cell populations within the cell capture channel. By selecting an appropriate shear stress to leukocyte cells bound to a cell capture channel, a first leukocyte cell population can be retained bound to the binding moiety within the cell capture channel, while a second leukocyte cell population can be removed from the cell capture channel. By applying a shear stress with a force on the second bound leukocyte cell population that is greater than the binding energy of the second leukocyte cell to the binding moiety, the second bound leukocyte cell population is released and flows out of the cell capture channel while cells of the first leukocyte population are retained within the cell capture channel. The first leukocytes can be retained as a result of experiencing a lower shear stress force due to having a different size from the second leukocytes, and/or a higher affinity for the binding moiety. The step of allowing the desired cells to bind to the binding moiety and the step of applying a shear stress, can occur simultaneously.

The cell capture channels (110, 140, 160) can be configured to capture neutrophils, lymphocytes and monocytes on a single substrate/chip, as shown in FIG. 1A. Each cell capture channel (140, 160, 180) can be coated with binding moieties for one or more types of leukocyte cells, and can have fluid flow paths of differing cross-sectional areas to provide different shear forces on bound leukocytes within each cell capture chamber. The first cell capture channel 140 can include a binding moiety attached to a fluid contacting wall that can bind both a first leukocyte of a first size and a second leukocyte of a second size. The first cell capture channel 140 can be configured to provide pass the leukocyte enriched fluid stream containing both the first and second leukocyte through the first cell capture channel 140 at a rate that imparts a shear stress to disrupt binding of the second leukocyte to the binding moiety, without substantially disrupting the binding of the first leukocyte to the binding moiety. The shear stress applied to the bound leukocyte can be estimated by the equation:

$$τ = \frac{6 Q η}{{w}^{2} h} \frac{1}{\{ 1 - 0.63 ( w / h ) \}}$$

where h is fluid viscosity, Q is volumetric flow rate, w is the channel width and h is the channel height.

Table 1 (below) provides exemplary cell populations, cell surface markers appropriate for the methods and devices of the invention, and the corresponding shear stresses necessary to isolate the indicated cells from a blood sample.

TABLE 1

| | Capture Shear | Wash | Capture | | |
|---|---|---|---|---|---|
| Cell-Type | [dynes/cm²] | [dynes/cm²] | Molecule | Purity* | Yield |
| Neutrophil | 0.3-0.5 | 1.5-2.0 | Anti-CD66b | >99 | 80% |
| Monocyte | 0.6-0.8 | 2.25-3.0 | Anti-CD14 | 91% | |
| | | | Anti-CD33 | | |
| | | | Anti-CD36 | | |
| Lymphocyte | 1.3-1.8 | 2.5-3 | Anti-CD2 | 95% | 80% |
| | | | Anti-CD3 | | |
| | | | Anti-CD4 | | |
| | | | Anti-CD8 | | |
| Neutrophils | 1-7 | — | E, P Selectins | 70% | 80% |
| HIV Specific | 0.082 | N/A | HLA A2-SL9 | >99% | N/A |
| T-cells | | | pentamer | | |
| Any-disease | 0.07-0.1 | | Pentamer | | |
| specific T-cell | | | | | |

*blood from healthy donor;
#blood from patients with cancer stage III-V

In this manner, a first leukocyte depleted stream is formed within the first cell capture channel 140 as the first leukocytes are retained therein, bound to the binding moiety, while binding of other leukocyte cell types to the binding moieties in the first cell capture channel are disrupted to permit these other leukocytes to pass through the first cell capture channel. Examples of suitable cell capture channels and shear stresses are provided in PCT patent application PCT/US2007/006791, filed Mar. 15, 2007 (published as WO2007/106598A2), which is incorporated herein by reference in its entirety.

Examples of suitable binding moieties include CD66b antibody for capture of neutrophils, CD14, CD33, and CD36 antibodies for capture of monocytes and CD2, CD4 antibodies, and CD2, CD3, CD4, and CD8 antibodies for capture of lymphocytes. Each cell capture channel (140, 160, 180) can be formed as a series of parallel microcapillary channels connected at common inlet and outlet points. For example, each of the cell capture channels (140, 160, 180) includes four parallel microcapillary channels (142, 162, 182, respectively) joined at common inlets and outlets.

The first cell capture channel 140 can be configured for neutrophil capture by coating the cell capture channel 140 with CD66b antibody using standard surface functionalization techniques (e.g., as described in the Examples herein), and applying an appropriate shear stress to remove other sample components that bind to this antibody. A suitable shear stress permitting retention of neutrophils captured on the CD66b antibody is 0.3 to 0.5, e.g., 0.40 to 0.45, or 0.45 dynes/cm². This shear force can be achieved in first cell capture microcapillary 142, for example, at a fluid flow rate of about 210 microliters/min through four parallel microcapillaries 142 each having a height of 250 μm, a width of 3.138 mm, a total width of the array of microcapillaries 142 (including space between channels) of 14.05 mm and a length of device of 43.75 mm. In this manner, at least a portion of a first leukocyte cell population can be retained within the first cell capture channel 140 as the leukocyte enriched stream passes through the first cell capture channel 140 and a first leukocyte depleted stream exits the first cell capture channel 140 at the outlet 144.

The second cell capture channel 160 can be configured for monocyte capture by coating the second cell capture channel 160 with CD36 antibody, and applying an appropriate shear stress to remove other sample components that bind to this antibody. A suitable shear stress permitting retention of monocytes captured on the CD36 antibody is 0.6 to 0.8, e.g., 0.7 to 0.75, dynes/cm². This shear force can be achieved in first cell capture microcapillary 162, for example, at a fluid flow rate of about 210 microliters/min through four parallel microcapillaries 162 each having a height of 250 μm, a width of 2.087 mm, a total width of the device (including space between channels) of 9.848 mm and a length of the array of microcapillaries of 63.89 mm. In this manner, at least a portion of a second leukocyte cell population can be retained within the second cell capture channel 140 as the first leukocyte depleted stream (e.g., a neutrophil-depleted stream) passes through the second cell capture channel 160 and a second leukocyte depleted stream exits the first cell capture channel 160 at the outlet 168.

The third cell capture channel 180 can be configured for lymphocyte capture by coating the second cell capture channel 180 with CD2 antibody, and applying an appropriate shear stress to remove other sample components that bind to this antibody. A suitable shear stress permitting retention of lymphocytes captured on the CD2 antibody is 1.3 to 1.8, e.g., 1.2 or 1.7 or 1.75, dynes/cm². This shear force can be achieved in first cell capture microcapillary 182, for example, at a fluid flow rate of about 210 microliters/min through four parallel microcapillaries 182 each having a height of 250 μm, a width of 1.06 mm, a total width of the device (including space between channels) of 5.74 mm and a length of the array of microcapillaries of 66.6 mm. In this manner, at least a portion of a third leukocyte cell population (e.g., lymphocytes) can be retained within the third cell capture channel 180 as the second leukocyte depleted stream (e.g., a neutrophil- and monocyte-depleted stream) passes through the third cell capture channel 180 and a third leukocyte depleted stream exits the first cell capture channel 160 at the outlet 168.

Monocytes can be challenging to capture within a cell capture channel compared to other leukocyte cell types, as a unique surface marker has not been identified for monocytes. CD14, CD33, and CD36 antibodies are all possible binding moieties and were contacted with a fluid sample containing monocytes, neutrophils, platelets, and lymphocytes. The CD14 antibody showed competition between soluble CD14 (sCD14) in serum and membrane CD14 (mCD14) on monocytes in whole blood, as well as low monocyte capture efficiency, and low expression levels on neutrophils (possible contamination). The CD33 antibody requires very low flow rates. Finally, the CD36 antibody can activate cells with time, and is expressed on platelets, but has a low capture efficiency for monocytes. In addition, we found that CD14 requires high antibody concentration.

For use with trauma and burn patients, another practical consideration is that CD14 and CD33 are reduced in expression in these patients. Thus, we have found that the CD36 antibody can be used to surprisingly good effect to isolate monocytes when the blood sample is initially depleted of platelets (which also express CD36) using the inertial focusing spiral microfluidic device, followed by cell capture and isolation in separate channels.

Leukocytes captured in each of the cell capture channels can be detected by lysing the cell contents of each cell capture channel and analyzing the cell lysate for each cell capture channel independently. Cell lysis can be done using similar protocol adopted by Glue Grant on a neutrophil chip for each cell capture channel. For example, after the unbound cells are washed off the integrated chip, a lysis buffer (e.g., Buffer RLT), is used to isolate nucleic acids (Qiagen, Hilden, Germany). The lysis buffer can be injected separately into each of the three chambers of the integrated device, e.g., using a blunt tip needle (Small Parts) attached to a 1 mL syringe (BD). The flow-through can be collected in a QIAShredder® column through Teflon® tubing attached to the outlet. The QIAShredder column is then centrifuged, the column discarded, and the lysate capped and stored at −80° C.

Lysis of bound leukocytes in one or more cell capture channels preferably provides an average of about 100 ng of RNA and a minimum of at least about 20 ng RNA from the lysed bound leukocyte cells within the cell capture channels. In addition, the cell capture channels can include s sufficient amount of protein for high throughput proteomics (HTP).

To quantify the purity, after the device is washed, the cells can be fixed by flowing in 1% paraformaldehyde. The cells can then be stained by flowing in a mixture of dye color solutions (see above). The cells can then be imaged and counted manually to gauge the purity. Purity count can be performed on a fluorescent microscope, by manually counting the imaged cells (e.g., fluorescently labeling to gauge purity count using CD36 PE (red), CD66b FITC (green), CD3 AF647 (cyan) and/or Hoechst (blue)). Preferably the cell capture channels isolate a single type of leukocyte with a purity of at least about 90% and cell quantity of around 10K-30K cells for all three cell leukocyte types.

In one particular method, a population of monocytes isolated in a cell capture channel is lysed within the cell capture channel, and the RNA collected from the lysate for downstream analysis. For example, the collected RNA can be used to perform RT-PCR using primers for the HLA-DR marker. HLA-DR has been widely discussed in medical literature to be able to predict the mortality of critically ill sepsis patients. The methods described herein can be methods of monitoring the HLA-DR using the integrated microfluidic cell capture devices described herein. These methods can not only predict the patients' health status, but can monitor the efficacy of the treatment regimen as well. This can be normalized and compared with the levels from a “universal donor.”

### EXAMPLES

The invention is further described in the following examples, which do not limit the scope of the invention described in the claims. Methods of making, analyzing, and characterizing some aspects of the invention are described below.

### Example 1

### Design and Microfabrication of the Integrated Device

A prototype integrated microfluidic device shown in FIG. 1A was fabricated using the prototyping techniques of soft lithography (Cheng, et al., “A microfluidic device for practical label-free CD4+ T cell counting of HIV-infected subjects,” Lab Chip, 2006, 6:170-178). In general, the channels were microfabricated using polydimethylsiloxane (PDMS), which in turn was irreversibly bonded to a glass slide through exposure to oxygen plasma (Cheng, et al., Lab Chip, 2006). The entire footprint of the chip was 75 mm by 50 mm.

Briefly, the layout of the integrated device was designed using AutoCAD software (AutoDesk, San Rafael, Calif., USA). Two high-resolution transparencies (Fineline Imaging, Colorado Springs, Colo., USA) were generated from the AutoCAD file and were used as the masks. The two layers of thicknesses of 50 μm and 250 μm were photolithographically patterned using photoresist SU8 (MicroChem, Newton, Mass., USA) on bare silicon wafers to make the negative masters. A PDMS base and the curing agent (Dow Corning, Midland, Mich., USA) were used in the ratio of 10:1 (wt/wt), mixed thoroughly, poured over the SU8 master, degassed for around 1 hour, and allowed to cure in a 65° C. oven for at least 8 hours.

The PDMS replicas were cut, removed from the surface of the master, followed by punching holes to provide the PDMS devices with the inlets and outlets. The PDMS replicas and glass slides (75 mm×50 mm×1 mm, Fisher Scientific, Fair Lawn, N.J., USA) were treated with oxygen plasma (100 mW, 1% oxygen, 30 seconds) in a PX-250 plasma etcher (March Instruments, Concord, Mass., USA) and then immediately placed in contact to irreversibly bond the two surfaces. Devices were baked at 75° C. on a hotplate for 5 minutes following bonding. Surface modification was carried out immediately after the baking step.

The channel dimensions (primarily width and height) for each capture region were designed to provide for the optimal capture shear stress for the respective cell type: neutrophils (0.45 dynes/cm²), monocytes (0.75 dynes/cm²) and lymphocytes (1.2 dynes/cm²). The arrays of high aspect ratio channels were designed, so as to be able to have a single flow rate through each of the capture regions.

The different capture regions were functionalized with their respective capture antibodies (CD66b for neutrophil, CD36 for monocytes, CD2 for lymphocytes) using the antibody coating technique (Cheng, et al., 2006). All surface functionalization solutions were introduced into the channels manually using syringes (BD Biosciences, San Jose, Calif., USA) with appropriately sized needle tips (Small Parts, Miramar, Fla., USA). Immediately after the oxygen plasma-bonding step, the capture channels of the integrated devices were flushed with a 5% (v/v) solution of 3-mercaptopropyl trimethoxysilane (3-MPS) [Gelest, Morrisville, Pa., USA] in 190-proof anhydrous ethanol (Sigma-Aldrich, St. Louis, Mo., USA), and allowed to react at room temperature for 30 minutes.

The unreacted silane was removed by flushing the device with ethanol, followed by which the device were baked at 80-90° C. till all the ethanol was evaporated from within the device. The devices were then treated with 1 mM of the coupling agent N-y-maleimidobutyryloxy succinimide ester (GMBS) (Pierce Biotechnology, Rockford, Ill., USA) in ethanol for around 30 minutes. Following this, the devices were flushed with deionized water until all the remaining ethanol inside the device was removed. The devices were then flushed with 50 μg/mL of NeutrAvidin (Pierce Biotechnology, Rockford, Ill., USA) in phosphate buffered saline (PBS) (10× stock obtained from Ambion, Austin, Tex., USA) and stored at 4° C. overnight. Before adding the antibody solution, the devices were flushed with ice-cold PBS solution containing 1% (w/v) bovine serum albumin (BSA) (lyophilized BSA was obtained from Aldrich Chemical Co., Milwaukee, Wis., USA) to remove any remaining unbound NeutrAvidin® molecules, as well as to provide for a non-specific surface.

Finally, the required antibody solutions were flown into their respective chamber, with the run-off being collected through tubing into a microfuge tube, so as to prevent contamination between the different chambers. In this work, 25 μg/mL of biotinylated anti-CD2 solution (Abd Serotec, Raleigh, N.C., USA), 20 μg/mL biotinylated anti-CD36 solution (LifeSpan Biosciences, Seattle, Wash., USA), and 5 μg/mL biotinylated anti-CD66b solution (Abd Serotec) were used. The antibody treated devices were left for either one hour at room temperature before performing the capture experiment, or overnight at 4° C. The devices were then washed with 1% BSA in PBS solution to remove the unbound antibody solutions.

The various components on the integrated device were connected to each other using external Tygon® tubing, which can be removed or inserted manually. This provides for collection of the cell lysate from each of the capture regions manually, without the possibility of contamination between cell types.

In our system, blood and buffer were propelled through the microfluidic devices with the aid of a programmable syringe pump.

### Example 2

### Cell Activation Analysis of Cells Flowing Through the Differential Inertial Focusing Spiral Device

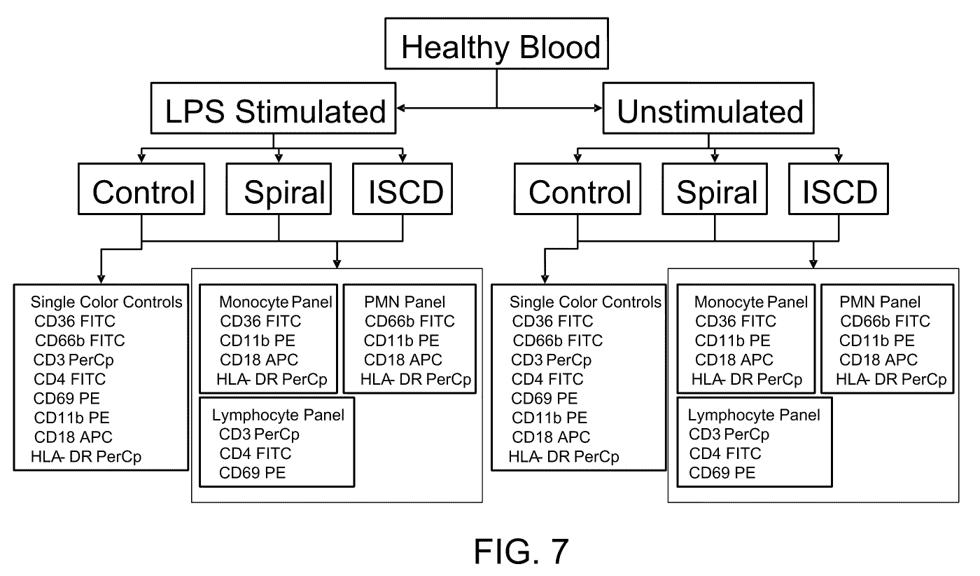

FIG. 7 is a flow chart of experiments performed to demonstrate that the flow rate used in Example 1 did not activate the leukocytes. It was also necessary to show that if the cells are activated, the spiral does not in turn change the activation status of the cells.

To test for possible activation of the cells, an experiment was conducted in which blood cells were activated using lipopolysaccharides (LPS) to have a positive case for reference. The activation markers that were probed using FACS (fluorescent activated cell sorting) for each of the three populations were as follows: neutrophils and monocytes with CD11b and CD18, HLA-DR, and lymphocytes with CD69. It was also necessary to test whether activated cells would down-regulate their activation levels when flowed through the spiral and capture device (note the integrated device is known as the integrated spiral capture device (ISCD) in the legend). Results are displayed for neutrophils, and it can be seen that two categories were always maintained. As shown non-activated as wells as the activated cells retain their initial state. A similar trend was seen for the monocytes and lymphocytes.

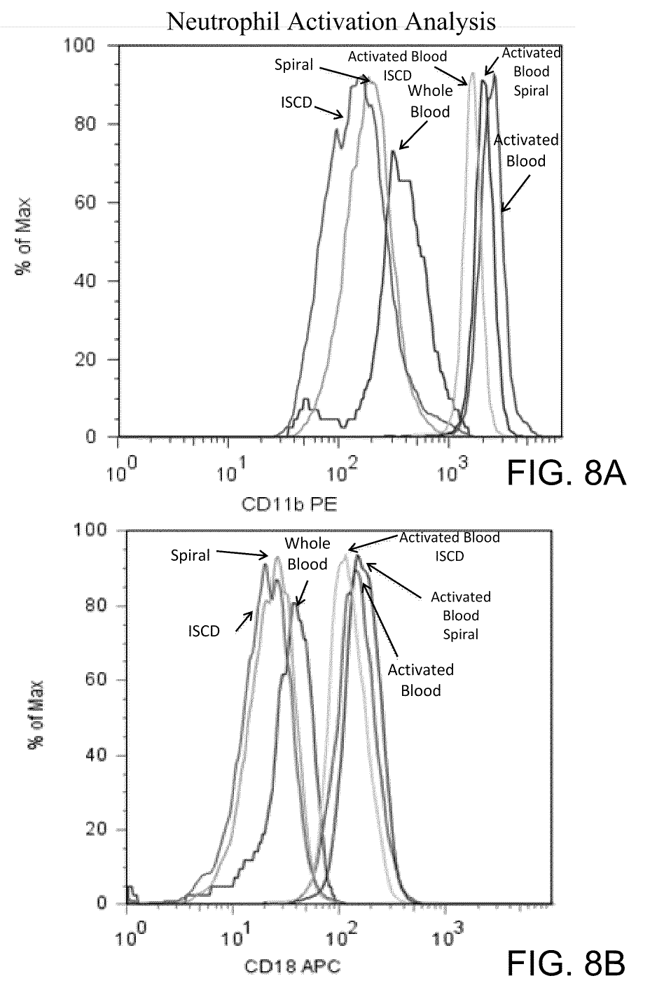

In particular, FIGS. 8A and 8B are images obtained from FACS (fluorescent activated cell sorting) of leukocytes and show the three leukocyte cell types (neutrophils, monocytes, lymphocytes) either in an activated state or not activated. In addition these graphs show that when leukocyte cells were activated using LPS, passing the leukocyte cells through the fluid flow path of the integrated microfluidic device in FIG. 1A did not alter the activation state of the leukocyte cells.

### Example 3

### Separation of Platelets and Leukocytes Using Differential Inertial Focusing in Curved, High Aspect Ratio Microfluidic Channels

In this work a spiral shaped device was used. Whole blood flown through the spiral device did not show appreciable separation between the platelets and leukocytes. Diluted blood was shown to have platelet and leukocyte separation. In this work, the spiral shaped device had two inputs, and two outputs, with the two outputs with different outlet amounts (outlet 2 to outlet 1 is in the ratio of 20:1 (w/w)). This was done so that the processed blood from outlet 1 would not be appreciably diluted after flowing through the spiral device.

Varying the flow rate of the buffer as well as the blood input flow rate attained on-chip blood dilution. For example, for 2% blood dilution the buffer was injected at 2450 microliters/min and blood was injected at 50 microliters/min. The leukocyte and platelet concentration from the two outlets were processed using a Sysmex® KX-21N complete blood analyzer. Initial results are shown in FIG. 6, with the plot showing the normalized average concentration for the two components of platelets and leukocytes (white blood cell count) for different cases. In our work, 2% blood dilution was chosen so as to have a balance between platelet contamination amount and an optimal blood flow rate in order to have a faster capture rate.

### Example 4

### Capture and Isolation of Total Leukocyte Subpopulations

To gauge the effectiveness of the operation of the integrated device, four parameters are used: (1) number of total cells captured, (2) purity of the target cell captured, (3) RNA quality, and (4) RNA quantity isolated. The cell capture efficiency was also estimated for each of the three cell types in their respective chambers. Peripheral blood from five healthy volunteers was used to characterize the performance of the integrated device. For each volunteer case, one device was used to assess the cell number and capture purity, while the second device was used to obtain the cell lysate for assessing the RNA quality and quantity.

To quantify the cell count and purity, the captured cells were fixed by flowing 1% (v/v) formaldehyde (prepared from paraformaldehyde (Electron Microscopy Sciences, Hatfield, Pa., USA) in 1×PBS and stored at 4° C. for future use), and incubating the solution for 20 minutes or longer at 4° C. The fixed cells were then rinsed with PBS, incubated with an antibody mixture containing fluorescein isothiocyanate (FITC) anti-CD66b, phycoerythrin (PE) anti-CD36, and Alexa Fluor 647 anti-CD3 (all from BD Biosciences, San Jose, Calif., USA), followed by Hoechst 33342 stain (Invitrogen, Carlsbad, Calif., USA), with all the dye solutions diluted in 1% BSA solution in 1×PBS, followed by being imaged on an inverted microscope (Nikon Eclipse TE2000, Nikon, Japan). All the capture chambers were treated with the same cocktail mixture of dyes and were imaged using the same UV, FITC, Cy3 and Cy5 excitation/emission filters.

The cells were counted manually using tools from ImageJ software (Rasband, W. S., ImageJ, U.S. National Institutes of Health, Bethesda, Md., USA, http://rsb.info.nih.gov/ij/, 1997-2009.). It should be pointed out that to avoid competitive binding between the capture and the labeling antibodies, they were all selected to be of different clones.

After the unbound cells were washed off the chip, to isolate the RNA, RLT lysis buffer solution (Qiagen, Hilden, Germany) was injected separately into each of the three chambers of the integrated device manually using a blunt tip needle (Small Parts) attached to a 1 mL syringe (BD). The flow-through was collected into a QIAShredder column through Teflon tubing attached to the outlet. The QIAShredder column was then centrifuged, the column discarded, and the lysate capped and stored at −80° C. Total RNA was isolated using the RNeasy Plus Mini Kit (Qiagen). The purified RNA was suspended in RNase-free water for downstream processing. To assess the quantity and the quality of RNA extracted from all the three chambers on the devices, we utilized the NanoDrop 3300 Fluorospectrometer (NanoDrop Technologies, Wilmington, Del., USA) and the Bioanalyzer 2100 (Agilent, Palo Alto, Calif., USA), respectively.

The summary of the integrated device performance results is presented in Table 2. The purity of the cells obtained for all the three cell subtypes (lymphocytes 89%±11%, monocytes 91%±7%, and neutrophils 96%±4%; an average of over 90%) is comparable to macroscale methods. The capture efficiencies (mean±standard deviation), estimated as total target cells captured divided by the total number of target cells flown in, of the various components are (3.1±2.4)%, (19.0±13.5)%, and (22.0±10.4)% for lymphocytes, neutrophils, and monocytes respectively. Specifically for the monocyte case, the platelet to leukocyte ratio was enumerated to be around (3.4±1.7) during the characterization of all the chips.

The RNA integrity number (RN) measures RNA quality and grades it on a quantitative scale of 1 (poor) to 10 (high). The quality of the isolated RNA (expressed as RIN) was consistently high (see Table 2), and the quantity of the isolated RNA was consistently greater than 5 ng. Hence, the 500 μL of processed blood produced RNA from each of the three captured sub-populations in amounts that are sufficient for downstream microarray analysis.

In comparison among all the three cell sub-types, the relative low parameter numbers for the lymphocytes can be accounted due to the fact that the surface area of the capture chamber was around one-half compared to that for the neutrophils and monocytes (channel 180 in FIG. 1A). The overall low capture efficiencies (<50%) of the three different cell sub-types can most likely be attributed to the height of the capture channels (250 μm) that are ˜30× times larger than the size of the typical leukocyte (˜8 μm). The transit time, tₜ, of the flowing cells across their respective capture channels (˜32 seconds for both neutrophils and monocytes, ˜17 seconds for lymphocytes) is much shorter (or in the same order as in the case of monocytes) than their respective cell settling time, tₛ: ˜35 seconds for monocytes, ˜62 seconds for neutrophils, and ˜160 seconds for lymphocytes).

This means that the cells do not have sufficient time to have contact with the capture area. It is interesting to note that (tₜ/4tₛ) gives non-dimensional percentage values (4% for lymphocytes, 19% for neutrophils, and 28% for monocytes) that are very close in the order of magnitude to the corresponding estimated capture efficiencies shown above.

TABLE 2

| | Cell | | | |
|---|---|---|---|---|
| | Number | RNA Quality | RNA | Concentration |
| Cell Type | (approx.) | (RIN) | Amount (ng) | (ng/mL) |
| Monocytes | 34,000 | 9.4 ± 0.8 | 13.5 ± 12.0 | 225 ± 192 |
| Neutrophils | 281,000 | 9.3 ± 0.3 | 11.9 ± 7.3 | 199 ± 122 |
| Lymphocytes | 26,000 | 8.2 ± 0.6 | 7.8 ± 3.0 | 129 ± 50 |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

We claim:

1. A method for capturing and detecting a leukocyte sub-population from a blood sample, the method comprising:
a. providing a sample comprising a first leukocyte cell population, a second leukocyte cell population and a platelet cell population;
b. passing the sample through a spiral channel to separate the sample into a platelet enriched stream and a leukocyte enriched stream;
c. passing the leukocyte enriched stream through a first cell capture channel at a first rate to capture and retain at least a portion of the first leukocyte cell population within the first cell capture channel, thereby reducing the first leukocyte cell population in the leukocyte enriched stream; and
d. detecting the first leukocyte cell population retained in the first cell capture channel.

2. The method of claim 1, further comprising
a. lysing at least a portion of the first leukocyte cell population retained in the first cell capture channel; and
b. detecting the first leukocyte cell population by measuring the amount RNA obtained from the first cell capture channel after lysing the portion of the retained first leukocyte cell population.

3. The method of claim 2, wherein the RNA quality, expressed as RNA integrity number (RIN), is at least about 7.5.

4. The method of claim 2, wherein the total amount of RNA is at least about 5.0 ng or greater.

5. The method of claim 1, wherein the first leukocyte cell population in the leukocyte enriched stream is reduced by at least about 90% while passing through the first cell capture channel.

6. The method of claim 1, wherein the first leukocyte cell population and the second leukocyte cell population are different types of leukocyte cells selected from the group consisting of: monocytes, neutrophils, and lymphocytes.

7. The method of claim 1, wherein the sample is passed through a spiral channel without altering the activation state of the first leukocyte cell population.

8. The method of claim 7, wherein the first leukocyte cell population is inactive.

9. The method of claim 1, wherein the first leukocyte cell population comprises a majority of neutrophils.

10. The method of claim 9, wherein the second leukocyte cell population comprises a majority of monocytes.

11. The method of claim 1, further comprising
a. passing a first leukocyte depleted stream obtained from the first cell capture channel through a second cell capture channel at a second rate to capture and retain at least a portion of the second leukocyte cell population within the second cell capture channel, thereby reducing the second leukocyte cell population in the first leukocyte depleted stream; and
b. detecting the second leukocyte cell population retained in the second cell capture channel.

12. The method of claim 11, wherein the first leukocyte population comprises a majority of neutrophils and the second leukocyte cell population comprises a majority of monocytes.

13. The method of claim 11, wherein the first rate and the second rate are substantially equal and the volume of the first cell capture channel is different from the volume of the second cell capture channel.

14. The method of claim 11, further comprising
a. passing a second leukocyte depleted stream obtained from the second cell capture channel through a third cell capture channel at a third rate to capture and retain at least a portion of a third leukocyte cell population within the third cell capture channel, reducing the third leukocyte cell population in the second leukocyte depleted stream; and
b. detecting the third leukocyte cell population retained in the third cell capture channel.

15. The method of claim 14, wherein the first leukocyte population comprises a majority of neutrophils, the second leukocyte cell population comprises a majority of monocytes and the third leukocyte population comprises a majority of lymphocytes.

16. A method for capturing multiple leukocyte sub-populations from a blood sample, the method comprising:
a. providing a sample comprising a first leukocyte cell, a second leukocyte cell, and a platelet cell population;
b. passing the sample through a spiral channel to separate the sample into a platelet enriched stream and a leukocyte enriched stream without altering the activation state of the first leukocyte cell; and
c. passing the leukocyte enriched stream through a first cell capture channel to remove the first leukocyte cell from the leukocyte enriched stream.

17. The method of claim 16, further comprising
a. retaining the first leukocyte cell within the first cell capture channel;
b. lysing the first leukocyte cell retained in the first cell capture channel; and
c. detecting the first leukocyte cell by measuring the amount of RNA obtained from the first cell capture channel after lysing the portion of the retained first leukocyte cell population.

18. The method of claim 16, wherein the leukocyte enriched stream further comprises a second leukocyte cell that is different from the first leukocyte cell; and wherein the first leukocyte cell and the second leukocyte cell are selected from the group consisting of: monocytes, neutrophils, and lymphocytes.

19. The method of claim 16, further comprising
a. passing a second leukocyte depleted stream obtained from the first cell capture channel through a second cell capture channel at a second rate to capture and retain a second leukocyte cell population within the second cell capture channel, thereby reducing the second leukocyte cell population in the first leukocyte depleted stream; and
b. detecting the second leukocyte cell population retained in the second cell capture channel.

20. A method for capturing and detecting multiple leukocyte sub-populations from a blood sample, the method comprising:
a. providing a blood sample comprising a first leukocyte cell population, a second leukocyte cell population, and a platelet cell population;
b. passing the sample through a spiral channel to separate the sample into a platelet enriched stream and a leukocyte enriched stream without altering the activation state of the first leukocyte cell population;
c. passing the leukocyte enriched stream through a first cell capture channel at a first rate to capture and retain at least a portion of the first leukocyte cell population within the first cell capture channel, thereby reducing the first leukocyte cell population in the leukocyte enriched stream;
d. detecting the first leukocyte cell population retained in the first cell capture channel;
e. passing a first leukocyte depleted stream obtained from the first cell capture channel through a second cell capture channel at a second rate to capture and retain at least a portion of the second leukocyte cell population within the second cell capture channel, thereby reducing the second leukocyte cell population in the first leukocyte depleted stream;
f. detecting the second leukocyte cell population retained in the second cell capture channel;
g. passing a second leukocyte depleted stream obtained from the second cell capture channel through a third cell capture channel at a third rate to capture and retain at least a portion of a third leukocyte cell population within the third cell capture channel, reducing the third leukocyte cell population in the second leukocyte depleted stream; and
h. detecting the third leukocyte cell population retained in the third cell capture channel.

21. The method of claim 20, wherein the first leukocyte population comprises a majority of neutrophils, the second leukocyte cell population comprises a majority of monocytes, and the third leukocyte population comprises a majority of lymphocytes.
